(19) United States
(12) Patent Application Publication
(10) Pub. No.: US 2008/0319218 A1
(43) Pub. Date: Dec. 25, 2008

(54) Processes for Making and Using Benzyl Pentahydroxyhexylcarbamoylundecanoate

(76) Inventors: Andreas Haubrich, Frankfurt (DE); Bernd Junker, Frankfurt (DE); Javier Manero, Frankfurt (DE); Theo Wollmann, Frankfurt (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/767,284

(22) Filed: Jun. 22, 2007

(51) Int. Cl. C07C 69/96; C07C 227/00; C07C 227/04; C07C 69/66

(52) U.S. Cl. 558/275; 560/170; 560/61; 562/587

(57) ABSTRACT

Provided are compounds and a process for preparing benzyl pentahydroxyhexylcarbamoylundecanoate (I) and uses thereof.

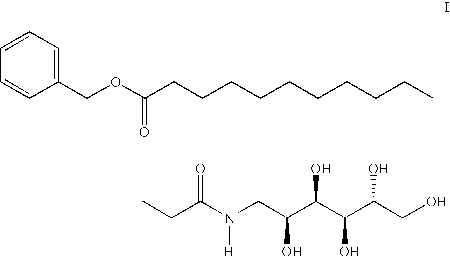

Provided are certain processes for the preparation of benzyl pentahydroxyhexylcarbamoylundecanoate and certain uses therefore.

Benzyl pentahydroxyhexylcarbamoylundecanoate has the formula I

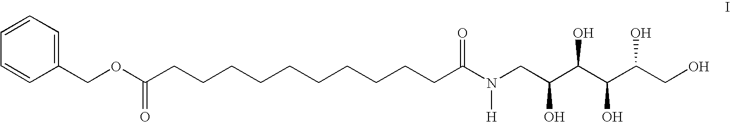

and is an intermediate in the preparation of the compound of formula II

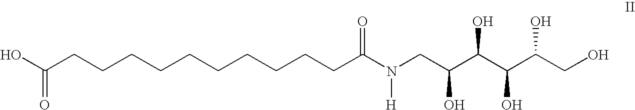

which in turn is an intermediate in the synthesis of the compound of formula III

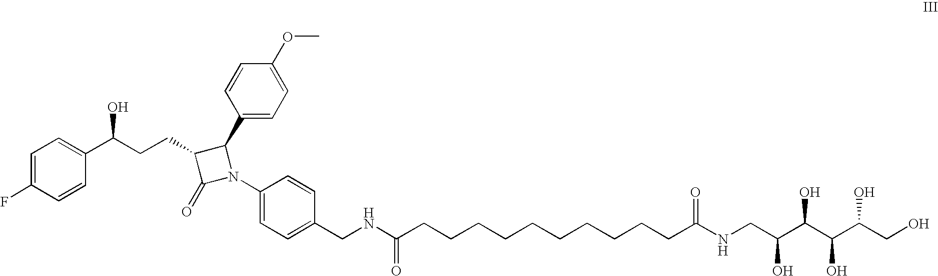

which is described in U.S. Pat. No. 7,205,290 as having, for example, cholesterol-lowering properties.

Provided is a process for preparing a compound of formula I

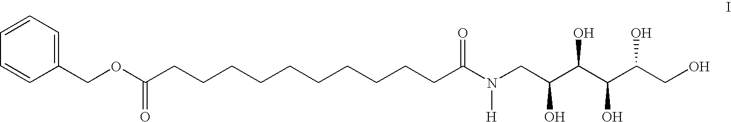

comprising
a) reacting a compound of formula IV

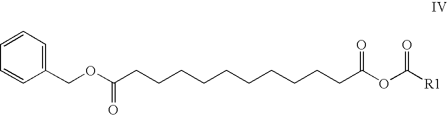

with a compound of formula VI

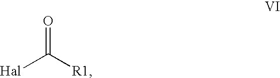

wherein
   Hal is chosen from Br, Cl, and I, and
   R1 is an alkyl radical which has from 1 to 18 carbon atoms and in which
      at least one —CH₂— group of the alkyl radical is optionally replaced by at least one group chosen from —O—, —CO—, —CH═CH—, —C≡C—, and aryl groups, and
      the alkyl radical is optionally substituted by at least one halogen chosen from F, Cl, Br, and I,
to form a compound of formula V

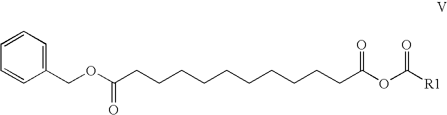

and
b) reacting the compound of formula V with D-glucamine to form the compound of formula I.

Also provided is a process for preparing a compound of formula I

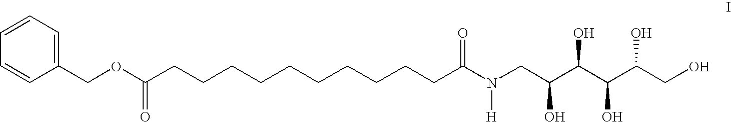

comprising
a) reacting a compound of formula IV

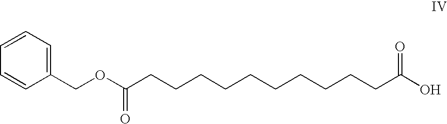

with a compound of formula VI

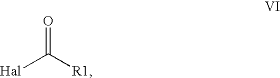

wherein Hal is chosen from Br, Cl, and I, and
   R1 is an alkyl radical which has from 1 to 18 carbon atoms and in which
      at least one —CH₂— group of the alkyl radical is optionally replaced by at least one group chosen from —O—, —CO—, —CH═CH—, —C≡C—, and aryl groups, and
      the alkyl radical is optionally substituted by at least one halogen chosen from F, Cl, Br, and I,
to form a compound of formula V

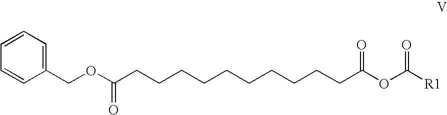

b) reacting the compound of formula V with monobenzyl ester of dodecanedioic acid of formula IV to form a compound of formula VIII

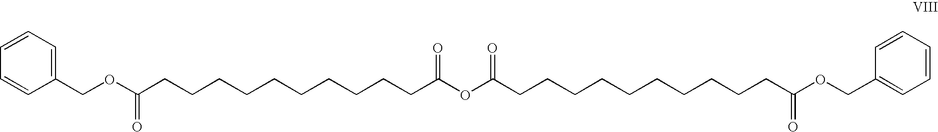

and
c) reacting the compound of formula VIII with D-glucamine to form the compound of formula I.

Also provided is a process of preparing a compound of formula I

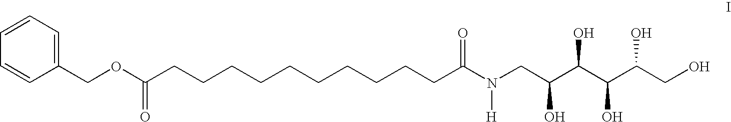

comprising converting a compound of formula V

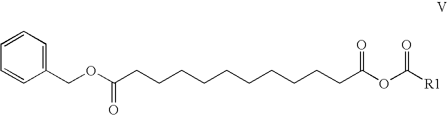

to the compound of formula I.

Also provided is a process of preparing a compound of formula I

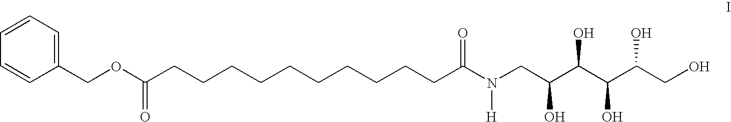

comprising converting a compound of formula Va

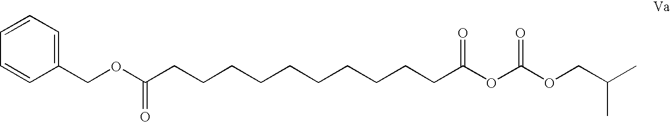

to the compound of formula I.

Also provided is a process for preparing a compound of formula Va

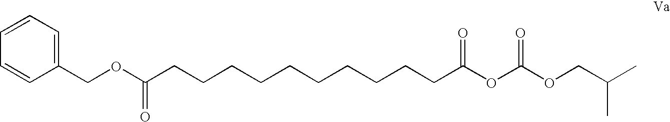

comprising reacting a compound of formula IV

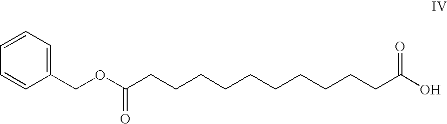

with a compound of formula VIa

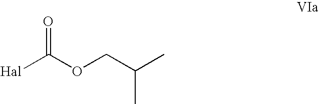

wherein Hal is chosen from Br, Cl, and I to form the compound of formula Va.

Also provided is a process for preparing a compound of formula I

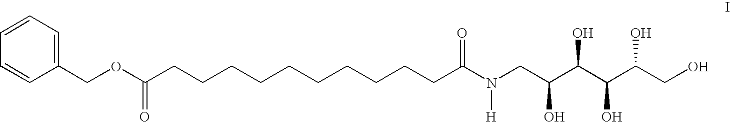

comprising reacting a compound of formula Va

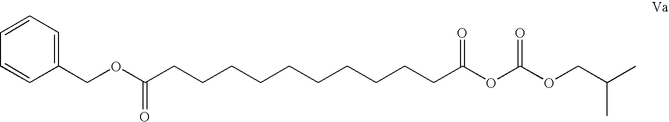

with D-glucamine to form the compound of formula I.

Also provided is a process for preparing a compound of formula VIII

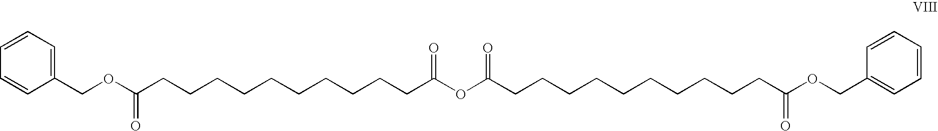

comprising
a) reacting a compound of formula IV

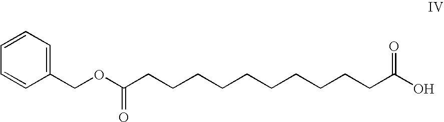

with a compound of formula VIa

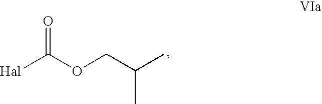

wherein Hal is chosen from Br, Cl, and I,
to form a compound of formula Va

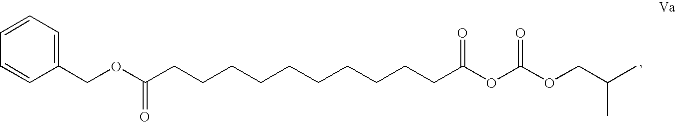

and
b) reacting the compound of formula Va with a compound of formula IV to form the compound of formula VIII.

Also provided is a process for preparing a compound of formula I

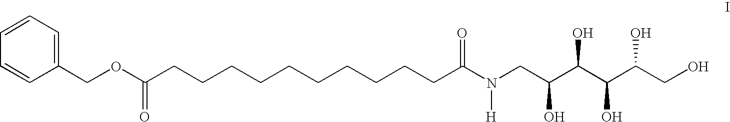

comprising reacting a compound of formula VIII

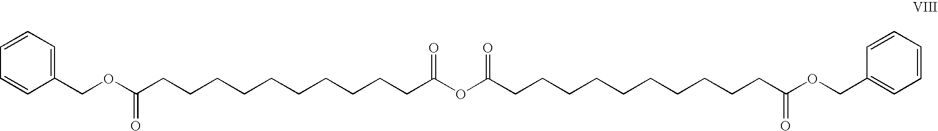

with D-glucamine to form the compound of formula I.

Also provided is a compound of formula V

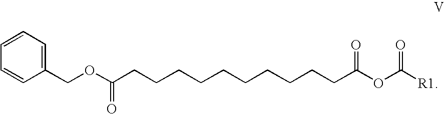

Also provided is a compound of formula Va

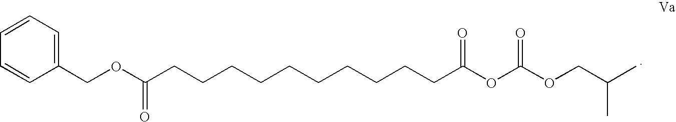

Also provided is a compound of formula VIII

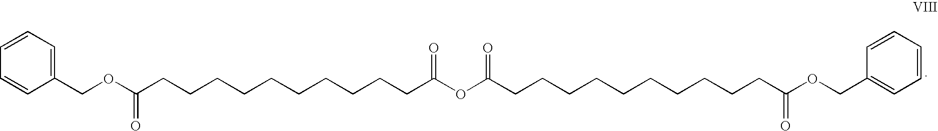

Other aspects and embodiments will be apparent to those skilled in the art from the following detailed description.

As used herein, the following words and phrases are generally intended to have the meanings as set forth below, except to the extent that the context in which they are used indicates otherwise.

As used herein, an alkyl radical is a straight-chain or branched hydrocarbon chain having from one to eighteen carbon atoms, e.g. methyl, ethyl, isopropyl, n-butyl, isobutyl, tert-butyl, hexyl, heptyl, octyl.

As used herein, an aryl radical is a phenyl, naphthyl, or biphenyl radical in which at least one CH group is optionally replaced by O, N, or S. The aryl radicals are optionally substituted by at least one suitable group, e.g.: F, Cl, Br, I, CF₃, NO₂, CN, COO(C₁-C₆)alkyl, CON[(C₁-C₆)alkyl]₂, cycloalkyl, (C₁-C₁₀)-alkyl, (C₂-C₆)-alkenyl, (C₂-C₆)-alkynyl, O—(C₁-C₆)-alkyl, O—(C₂-C₆)-alkenyl, O—(C₂-C₆)-alkynyl, O—CO—(C₁-C₆)-alkyl, O—CO—(C₁-C₆)-aryl, O—CO—(C₁-C₆)-heterocycle, SO₂N[(C₁-C₆)-alkyl]₂, S—(C₁-C₆)-alkyl, N((C₁-C₆)-alkyl)₂.

As used herein, the term “reacting” is intended to represent bringing the chemical reactants together under conditions such as to cause the chemical reaction indicated to take place.

As use herein, the term “converting” is intended to represent changing one compound into another, for example converting a compound of formula I into a compound of formula II.

The compounds described herein may be present in crystalline or amorphous solid forms. Those crystalline forms may include polymorphs and solvates, such as hydrates.

Provided is a process for preparing a compound of formula I

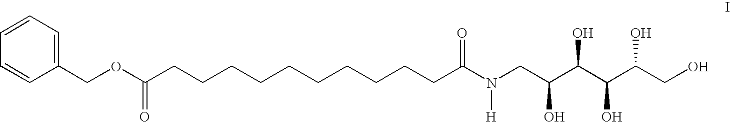

comprising
a) reacting a compound of formula IV

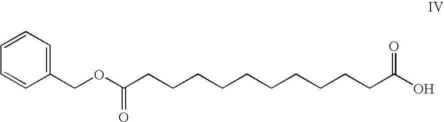

with a compound of formula VI

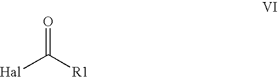

wherein
   Hal is chosen from Br, Cl, and I, and
   R1 is an alkyl radical which has from 1 to 18 carbon atoms and in which
      at least one —CH₂— group of the alkyl radical is optionally replaced by at least one group chosen from —O—, —CO—, —CH═CH—, —C≡C—, and aryl groups, and
      the alkyl radical is optionally substituted by at least one halogen chosen from F, Cl, Br, and I,
to form a compound of formula V

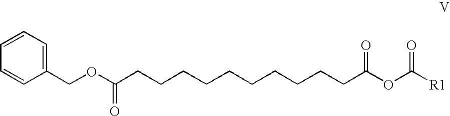

and
b) reacting the compound of formula V with D-glucamine to form the compound of formula I.

In some exemplary embodiments, the compound of formula VI is chosen from alkylcarboxylic halides and alkyl haloformates. In some exemplary embodiments, the compound of formula VI is isobutyl chloroformate.

In some exemplary embodiments, Hal is Cl.

In some exemplary embodiments, R1 is chosen from methyl, ethyl, propyl, isopropyl, butyl, isobutyl, tert-butyl, methyloxy, ethyloxy, propyloxy, isopropyloxy, butyloxy, isobutyloxy, tert-butyloxy, and benzyloxy. In some exemplary embodiments, R1 is isobutyloxy.

In some exemplary embodiments, in step a), the compound of formula IV is dissolved in a suitable solvent or solvent mixture in the presence of a suitable base at from −30° C. to 70° C., such as from −10° C. to 40° C., further such as from −5° C. to 0° C. A compound of formula IV may be added over a period of 30-150 minutes, such as 60-120 minutes, to a solution of a compound of formula VI. In some exemplary embodiments, the solution of a compound of formula VI may be cooled to from −10° C. to 30° C., such as from −10 to 0° C.

In some exemplary embodiments, in step a), a solution of a compound of formula VI in a suitable solvent or solvent mixture which is cooled to from −10° C. to 30° C., such as from −10° C. to 0° C., is added to a compound of formula IV and a suitable base in a suitable solvent or solvent mixture at from −30° C. to 70° C., such as from −10° C. to 40° C., further such as from −5° C. to 0° C., over a period of 30-150 minutes, such as 60-120 minutes.

In some exemplary embodiments, in step a), the reaction mixture is stirred at from −10° C. to 40° C., such as from −10° C. to 0° C., for from 15-150 minutes, such as 30-120 minutes. The reaction mixture may then either be used directly in the subsequent reaction or the product formed is isolated. In some exemplary embodiments, the reaction mixture is used directly. In some exemplary embodiments, the compound of formula V is isolated by evaporation of the solvent(s) under reduced pressure. In some exemplary embodiments, the reaction mixture is washed with water before the evaporation.

In some exemplary embodiments, the suitable base used in step a) is chosen from tertiary amines such as triethylamine, ethyldimethylamine, ethyldiisopropylamine, tributylamine, N-ethylmorpholine, tetramethylethylenediamine, guanidine, and alkyl guanidines. In some exemplary embodiments, the suitable base is chosen from triethylamine and ethyldiisopropylamine.

In some exemplary embodiments, the suitable solvent used in step a) is chosen from aprotic organic solvents such as toluene, chlorobenzene, dichloromethane, ethyl acetate, butyl acetate, diisobutyl ether, diisopropyl ether, tetrahydrofuran, 2-methyltetrahydrofuran, dimethylformamide, N-methylpyrrolidone, and methyl ethyl ketone. In some exemplary embodiments, the suitable solvent is chosen from ethyl acetate and butyl acetate. In some exemplary embodiments, a mixture of solvents is used.

In some exemplary embodiments, in step b), D-glucamine is added a little at a time over a period of from 5-60 minutes, such as 15-30 minutes, to a solution of a compound of formula V and a suitable base in a suitable solvent or solvent mixture at from −10° C. to 40° C., such as from −5° C. to 0° C.

In some exemplary embodiments, the suitable base used in step b) is chosen from tertiary amines such as triethylamine, ethyldimethylamine, ethyldiisopropylamine, tributylamine, N-ethylmorpholine, tetramethylethylenediamine, guanidine, and alkyl guanidines. In some exemplary embodiments, the suitable base is chosen from triethylamine and ethyldiisopropylamine.

In some exemplary embodiments, the suitable solvent used in step b) is chosen from aprotic organic solvents such as toluene, chlorobenzene, dichloromethane, ethyl acetate, butyl acetate, diisobutyl ether, diisopropyl ether, tetrahydrofuran, 2-methyltetrahydrofuran, dimethylformamide, N-methylpyrrolidone, and methyl ethyl ketone. In some exemplary embodiments, the suitable solvent is chosen from ethyl acetate and butyl acetate. In some exemplary embodiments, a mixture of solvents is used.

In some exemplary embodiments, the reaction mixture of step b) is stirred for a further period of from 5-120 minutes, such as 30-60 minutes, at from −10° C. to 40° C., such as from −5° C. to 0° C., subsequently for a further 5-20 hours, such as 12 hours, at from 0 to 30° C., such as from 15° C. to 20° C., and subsequently washed with water at 10° C. to 80° C., such as 50° C. to 70° C., and further such as 60° C. The mixture is subsequently cooled to a temperature sufficient to induce crystallization of the compound of formula I, such as 20° C. In some exemplary embodiments, the compound of formula I is purified by recrystallization.

Also provided is a process for preparing a compound of formula I

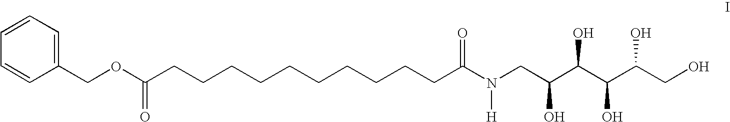

comprising
a) reacting a compound of formula IV

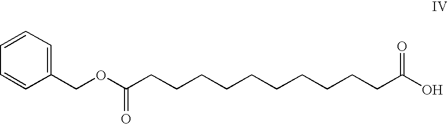

with a compound of formula VI

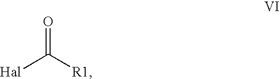

wherein Hal is chosen from Br, Cl, and I, and
R1 is an alkyl radical which has from 1 to 18 carbon atoms and in which
   at least one —CH₂— group of the alkyl radical is optionally replaced by at least one group chosen from —O—, —CO—, —CH═CH—, —C≡C—, and aryl groups, and
   the alkyl radical is optionally substituted by at least one halogen chosen from F, Cl, Br, and I,
to form a compound of formula V

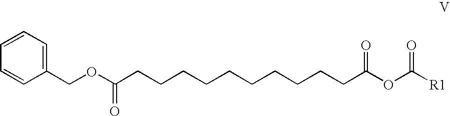

b) reacting the compound of formula V with the compound of formula IV to form a compound of formula VIII

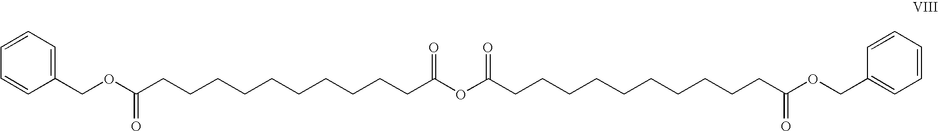

and
c) reacting the compound of formula VIII with D-glucamine to form the compound of formula I.

In some exemplary embodiments, the compound of formula VI is chosen from alkylcarboxylic halides and alkyl haloformates. In some exemplary embodiments, the compound of formula VI is isobutyl chloroformate.

In some exemplary embodiments, Hal is Cl.

In some exemplary embodiments, R1 is chosen from methyl, ethyl, propyl, isopropyl, butyl, isobutyl, tert-butyl, methyloxy, ethyloxy, propyloxy, isopropyloxy, butyloxy, isobutyloxy, tert-butyloxy, and benzyloxy. In some exemplary embodiments, R1 is isobutyloxy.

In some exemplary embodiments, in step a), the compound of formula IV is dissolved in a suitable solvent or solvent mixture in the presence of at least one base at from −30° C. to 70° C., such as from −10° C. to 40° C., further such as from −5° C. to 0° C., and added over a period of 30-150 minutes, such as 60-120 minutes, to a solution of compound of formula VI which is cooled to from −10° C. to 30° C., such as from −10 to 0° C.

In some exemplary embodiments, in step a), a solution of a compound of formula VI in a suitable solvent or solvent mixture is cooled to from −10° C. to 30° C., such as from −10° C. to 0° C., in a reaction vessel and then is added to a compound of formula IV and a suitable base in a suitable solvent or solvent mixture at from −30° C. to 70° C., such as from −10° C. to 40° C., further such as from −5° C. to 0° C., over a period of 30-150 minutes, such as 60-120 minutes.

In some exemplary embodiments, the reaction mixture in step a) is stirred at from −10° C. to 40° C., such as from −10° C. to 0° C., for from 15-150 minutes, such as 30-120 minutes. The reaction mixture can then either be used directly in the subsequent reaction or the product formed is isolated. In some exemplary embodiments, the reaction mixture is used directly. In some exemplary embodiments, the compound of formula V is isolated by evaporation under reduced pressure. In some exemplary embodiments, the reaction mixture is washed with water before the evaporation.

In some exemplary embodiments, the suitable base used in step a) is chosen from tertiary amines such as triethylamine, ethyldimethylamine, ethyldiisopropylamine, tributylamine, N-ethylmorpholine, tetramethylethylenediamine, guanidine, and alkyl guanidines. In some exemplary embodiments, the suitable base is chosen from triethylamine and ethyldiisopropylamine.

In some exemplary embodiments, the suitable solvent used in step a) is chosen from customary organic solvents such as toluene, chlorobenzene, dichloromethane, ethyl acetate, butyl acetate, diisobutyl ether, diisopropyl ether, tetrahydrofuran, 2-methyltetrahydrofuran, dimethylformamide, N-methylpyrrolidone, or methyl ethyl ketone. In some exemplary embodiments, the suitable solvent is ethyl acetate or butyl acetate. In some exemplary embodiments, a mixture of solvents is used.

In some exemplary embodiments, in step b), the compound of formula IV is added a little at a time to a solution of product V and optionally a suitable base in a suitable solvent or solvent mixture at from −10° C. to 40° C., such as from 0° C. to 25° C., over a period of 5-60 minutes, such as 15-30 minutes.

In some exemplary embodiments, the suitable base used in step b) is chosen from tertiary amines such as triethylamine, ethyldimethylamine, ethyldiisopropylamine, tributylamine, N-ethylmorpholine, tetramethylethylenediamine, guanidine, and alkyl guanidines. In some exemplary embodiments, the suitable base is chosen from triethylamine and ethyldiisopropylamine.

In some exemplary embodiments, the suitable solvent used in step b) is chosen from aprotic organic solvents such as toluene, chlorobenzene, dichloromethane, ethyl acetate, butyl acetate, diisobutyl ether, diisopropyl ether, tetrahydrofuran, 2-methyltetrahydrofuran, dimethylformamide, N-methylpyrrolidone, and methyl ethyl ketone. In some exemplary embodiments, the suitable solvent is chosen from ethyl acetate and butyl acetate. In some exemplary embodiments, a mixture of solvents is used.

In some exemplary embodiments, the reaction mixture of step b) is stirred at from −10° C. to 40° C., such as from 0° C. to 25° C., for another 5-240 minutes, such as 60-150 minutes. In some exemplary embodiments, the precipitate formed is filtered and dried, giving a compound of formula VIII.

In some exemplary embodiments, in step c), D-glucamine is added a little at a time to a solution of product VIII and optionally a suitable base in a suitable solvent or solvent mixture at from −10° C. to 40° C., such as from −5° C. to 5° C., over a period of from 5-60 minutes, such as 15-30 minutes.

In some exemplary embodiments, the suitable base used in step c) is chosen from tertiary amines such as triethylamine, ethyldimethylamine, ethyldiisopropylamine, tributylamine, N-ethylmorpholine, tetramethylethylenediamine, guanidine, and alkyl guanidines. In some exemplary embodiments, the suitable base is chosen from triethylamine and ethyldiisopropylamine.

In some exemplary embodiments, the suitable solvent used in step c) is chosen from aprotic organic solvents such as toluene, chlorobenzene, dichloromethane, ethyl acetate, butyl acetate, diisobutyl ether, diisopropyl ether, tetrahydrofuran, 2-methyltetrahydrofuran, dimethylformamide, N-methylpyrrolidone, and methyl ethyl ketone. In some exemplary embodiments, the suitable solvent is chosen from ethyl acetate and butyl acetate. In some exemplary embodiments, a mixture of solvents is used.

In some exemplary embodiments, the reaction mixture in step c) is stirred at from −10° C. to 40° C., such as from 10° C. to 25° C., for another 1-20 hours, such as 10-18 hours, and subsequently washed with water at 10° C. to 80° C., such as 50° C. to 70° C. The mixture is subsequently cooled to a temperature sufficient to induce crystallization of the compound of formula I, such as 20° C. In some exemplary embodiments, the compound of formula I is purified by recrystallization.

In some exemplary embodiments, the compound of formula I, which may be obtained as described herein, is converted into a compound of formula II

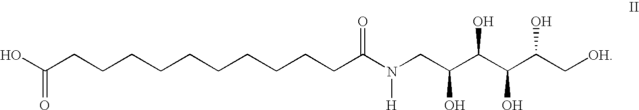

In some exemplary embodiments, converting the compound of formula I into a compound of formula II comprises reacting the compound of formula I under alkaline conditions, using, for example, aqueous sodium hydroxide or aqueous potassium hydroxide.

In some exemplary embodiments, converting the compound of formula I into a compound of formula II comprises reacting the compound of formula I under enzymatic conditions to form formula II. Non-limiting examples of suitable enzymes include lipases, for example Candida.

In some exemplary embodiments, converting the compound of formula I into a compound of formula II comprises hydrogenating the compound of formula I under suitable conditions.

In some exemplary embodiments, hydrogenating the compound of formula I comprises reacting the compound of formula I with a hydrogen source in the presence of a hydrogenation catalyst. In some exemplary embodiments, a hydrogenation catalyst, such as palladium on carbon (Pd/C), Raney-Nickel, platinum, platinum oxide, or zinc oxide is used. In some exemplary embodiments, the hydrogen source is chosen from hydrogen gas and ammonium formate.

Also provided is a process for preparing a compound of formula III

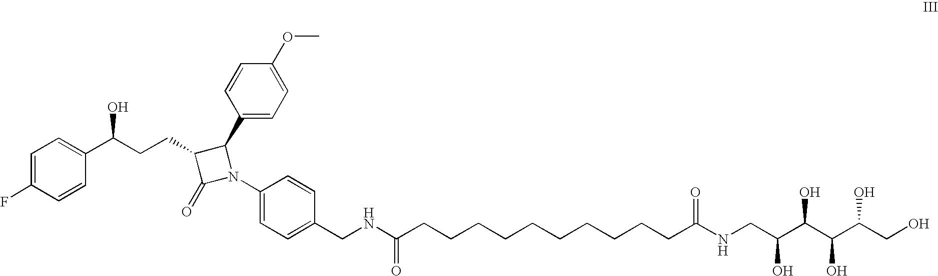

comprising
a) reacting a compound of formula IV

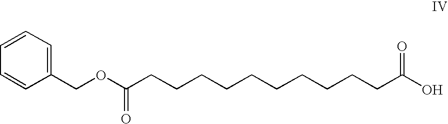

with a compound of formula VI

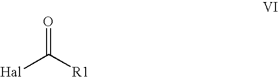

wherein Hal is chosen from Br, Cl, and I, and
R1 is an alkyl radical which has from 1 to 18 carbon atoms and in which
   at least one —CH₂— group of the alkyl radical is optionally replaced by at least one group chosen from —O—, —CO—, —CH═CH—, —C≡C—, and aryl groups, and
   the alkyl radical is optionally substituted by at least one halogen chosen from F, Cl, Br, and I,
to form a compound of formula V

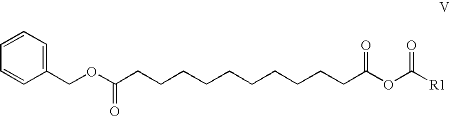

b) reacting the compound of formula V with D-glucamine to form the compound of formula I

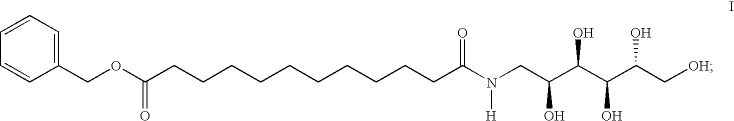

c) converting the compound of formula I into a compound of formula II

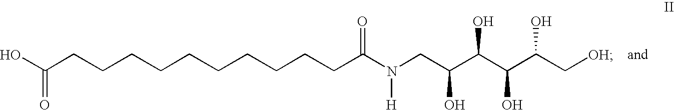

d) reacting the compound of formula II with a compound of formula VII

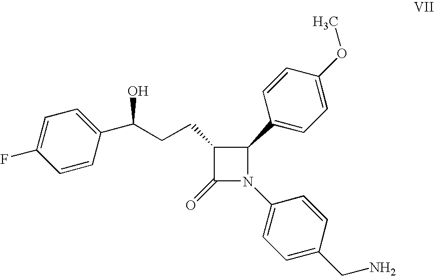

to form a compound of formula III.

In some exemplary embodiments, the reaction conditions for the preparation of a compound of formula V, a compound of formula I, and a compound of formula II are as described herein.

In some exemplary embodiments, reacting the compound of formula II with a compound of formula VII comprises reacting the compound of formula II with suitable peptide coupling reagents in a suitable solvent or solvent mixture, and then further reacting with a compound of formula VII. Suitable peptide coupling reagents and solvents or solvent mixtures are described, inter alia, in, for example, A. Speicher et al. In Journal für Praktische Chemie/Chemiker-Zeitung (1998), 340, 581-583; Y. S. Klausner and M. Bodansky, Synthesis, (1972), 453 et. seq.; K. Ishihara et al., J. Org. Chem., 61, 4196 (1996); M. Kunishima et al., Tetrahedron 55, 13159-13170 (1999), or R. C. Larock: Comprehensive Organic Transformations; VCH, New York, 1989, page 981 et. seq.

The reaction of the compound of formula II with the amine of formula VII is described, for example, in WO 02/50027 or U.S. Pat. No. 7,205,290.

Also provided herein is a process for preparing a compound of formula III

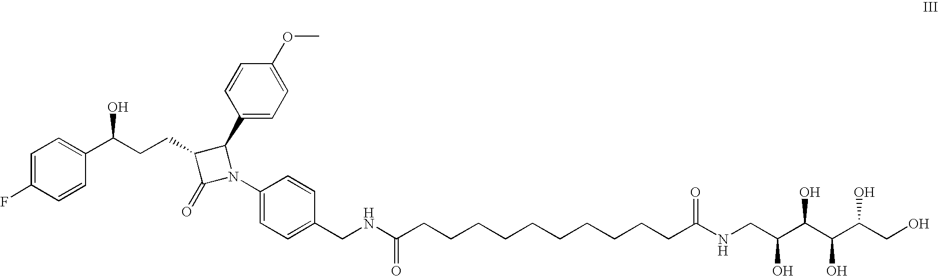

comprising
a) reacting a compound of formula IV

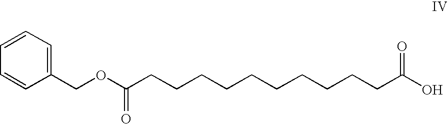

with a compound of formula VI

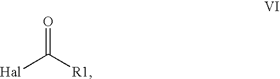

wherein Hal is chosen from Br, Cl, and I, and
R1 is an alkyl radical which has from 1 to 18 carbon atoms and in which
   at least one —CH₂— group of the alkyl radical is optionally replaced by at least one group chosen from —O—, —CO—, —CH═CH—, —C≡C—, and aryl groups, and
   the alkyl radical is optionally substituted by at least one halogen chosen from F, Cl, Br, and I,
to form a compound of formula V

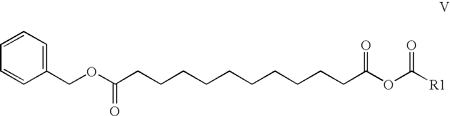

b) reacting the compound of formula V with monobenzyl ester of dodecanedioic acid of formula IV to form a compound of formula VIII

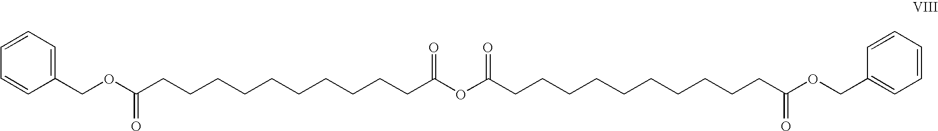

c) reacting the compound of formula VIII with D-glucamine to form the compound of formula I

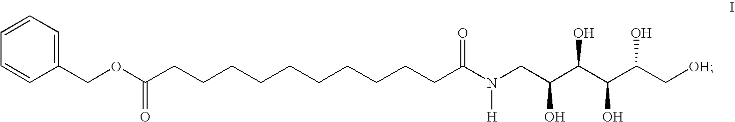

d) converting the compound of formula I into a compound of formula II

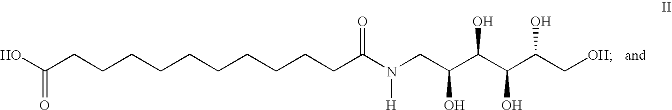

e) reacting the compound of formula II with a compound of formula VII

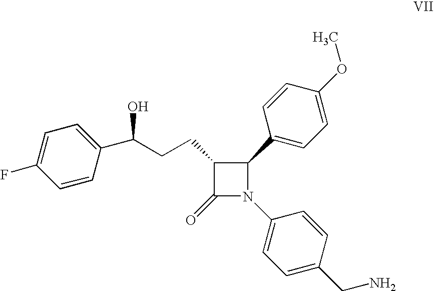

to form a compound of formula III.

In some exemplary embodiments, the reaction conditions for the preparation of a compound of formula V, a compound of formula VIII, a compound of formula I, a compound of formula II, and a compound of formula III are as described herein.

Also provided is a process of preparing a compound of formula I

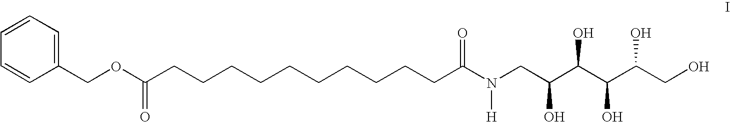

comprising converting a compound of formula V

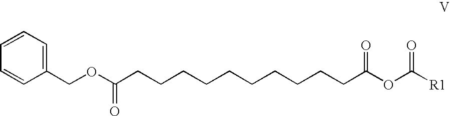

to the compound of formula I.

In some exemplary embodiments, the reaction conditions for the preparation of a compound of formula I from a compound of formula V are as described herein.

Also provided is a process of preparing a compound of formula I

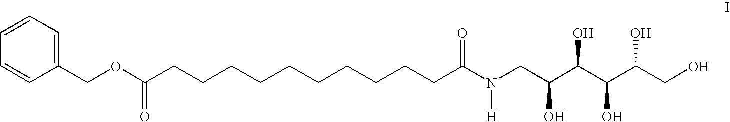

comprising converting a compound of formula Va

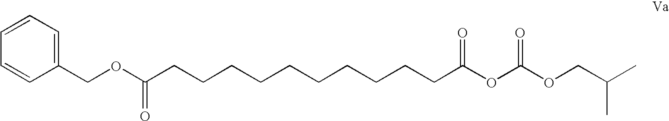

to the compound of formula I.

In some exemplary embodiments, the reaction conditions for the preparation of a compound of formula I from a compound of formula Va are as described herein.

Also provided is a process for preparing a compound of formula Va

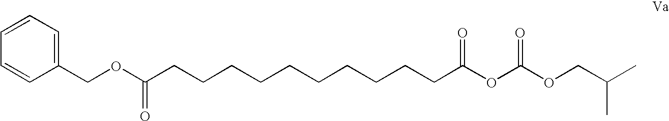

comprising reacting a compound of formula IV

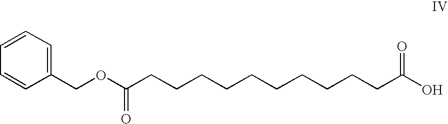

with a compound of formula Via

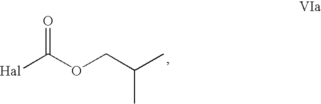

wherein Hal is chosen from Br, Cl, and I to form a compound of formula Va.

In some exemplary embodiments, the processes for the preparation of a compound of formula Va from a compound of formula IV are as described herein.

Also provided is a process for preparing a compound of formula I

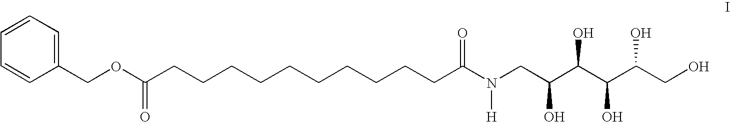

comprising reacting a compound of formula Va

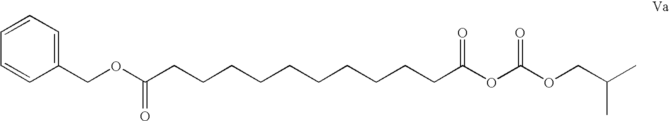

with D-glucamine to form the compound of formula I.

In some exemplary embodiments, the reaction conditions for the preparation of a compound of formula I from a compound of formula Va are as described herein.

Also provided is a process for preparing a compound of formula VIII

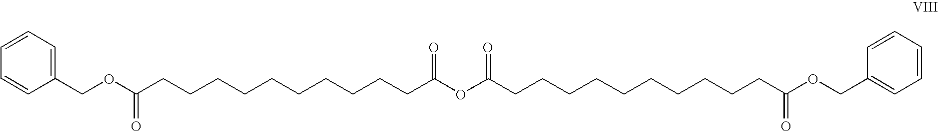

comprising
a) reacting a compound of formula IV

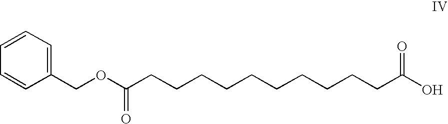

with a compound of formula VIa

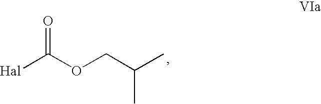

wherein Hal is chosen from Br, Cl, and I,
to form a compound of formula Va

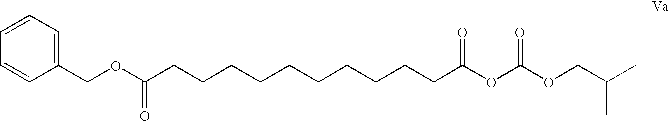

and,
b) reacting the compound of formula Va with the compound of formula IV to form the compound of formula VIII.

In some exemplary embodiments, the reaction conditions for the preparation of a compound of formula Va from a compound of formula IV and for the preparation of a compound of formula VIII from a compound of formula Va are as described herein.

Also provided is a process for preparing a compound of formula I

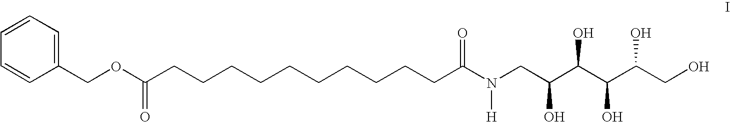

comprising reacting a compound of formula VIII

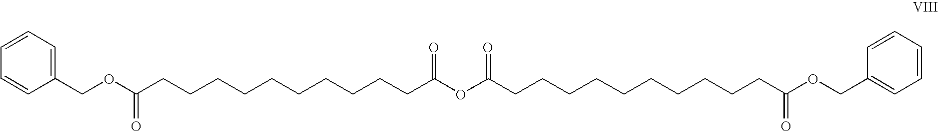

with D-glucamine to form the compound of formula I.

In some exemplary embodiments, the reaction conditions for the preparation of a compound of formula I from a compound of formula VIII are as described herein.

Also provided is a compound of formula Va.

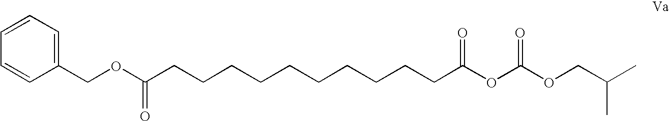

Also provided is a compound of formula VIII.

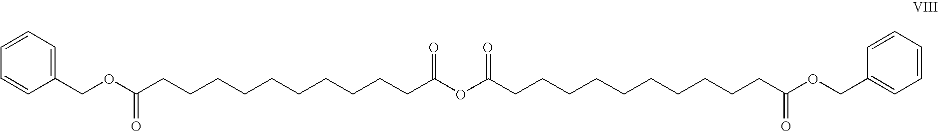

### EXAMPLES

The following examples serve to more fully describe the manner of using the invention. These examples are presented for illustrative purposes and should not serve to limit the true scope of the invention.

### 1. Preparation of benzyl 12-isobutoxycarbonyloxy-12-oxododecanoate Va

1.5 g (4.7 mmol) of the monobenzyl ester of dodecanedioic acid together with 15 ml of ethyl acetate are placed in a reaction vessel and admixed with 0.8 ml (5.6 mmol) of triethylamine. The mixture is cooled to −5° C. and a solution of 0.7 ml (5.0 mmol) of isobutyl chloroformate is added. After 60 minutes, the precipitate is filtered off with suction under protective gas, washed twice with ethyl acetate, and the filtrate is evaporated to dryness to obtain benzyl 12-isobutoxycarbonyloxy-12-oxododecanoate (Va).

### 2. Preparation of benzyl 11-(2S,3R,4R,5R-2,3,4,5,6-pentahydroxyhexylcarbamoyl)undecanoate I from benzyl 12-isobutoxycarbonyloxy-12-oxododecanoate (Va)

Benzyl 12-isobutoxycarbonyloxy-12-oxododecanoate is dissolved in 15 ml of ethyl acetate and admixed at 0° C. with 0.9 g (5.2 mmol) of 2R,3R,4R,5S-6-aminohexane-1,2,3,4,5-pentanol (D-glucamine). The mixture is stirred at 0° C. for one hour, warmed to 20° C. and allowed to stand overnight. The white suspension formed is shaken three times at 65° C. with 20 ml of water at the same temperature. The organic phase is subsequently evaporated to dryness to obtain benzyl 11-(2S,3R,4R,5R-2,3,4,5,6-pentahydroxyhexylcarbamoyl)undecanoate (I).

### 3. Preparation of the Anhydride of Dodecanedicarboxylic Monobenzyl Ester (VIII) from Benzyl 12-isobutoxycarbonyloxy-12-oxododecanoate (Va)

Benzyl 12-isobutoxycarbonyloxy-12-oxododecanoate is admixed with 20 ml of ethyl acetate and admixed at 20° C. with 1.9 g (4.2 mmol) of the monobenzyl ester of dodecanedioic acid. The mixture is stirred for 2.5 hours, and the precipitate formed is filtered off and dried to obtain anhydride of dodecanedicarboxylic monobenzyl ester (VIII).

### 4. Preparation of benzyl 11-(2S,3R,4R,5R-2,3,4,5,6-pentahydroxyhexylcarbamoyl)undecanoate (I) from the Anhydride of Dodecanedioic Acid Monobenzyl Ester (VIII)

0.50 g (0.80 mmol) of the anhydride of dodecanedioic acid monobenzyl ester (VIII) together with 10 ml of ethyl acetate are placed in a reaction vessel and admixed with 0.14 ml (0.96 mmol) of triethylamine and the mixture is cooled to 0° C. A suspension of 0.16 g (0.88 mmol) of 2R,3R,4R,5S-6-aminohexane-1,2,3,4,5-pentanol (D-glucamine) in 6 ml of ethyl acetate is added and the mixture is stirred at room temperature for 18 hours. The mixture is heated to 70° C. and shaken three times with 20 ml of water at the same temperature. The organic phase is allowed to cool to room temperature, and the precipitate formed is filtered off and dried to obtain benzyl 11-(2S,3R,4R,5R-2,3,4,5,6-pentahydroxyhexylcarbamoyl)undecanoate (I).

### 5. Preparation of benzyl 11-(2S,3R,4R,5R-2,3,4,5,6-pentahydroxyhexylcarbamoyl)undecanoate (I) from the Monobenzyl Ester of Dodecanedioic Acid (IVa) without Isolation of the Intermediates

17 kg of isobutyl chloroformate together with 150 L of ethyl acetate are placed in a reaction vessel and cooled to −5° C. A solution of 37.3 kg of the monobenzyl ester of dodecanedioic acid and 14.2 kg of triethylamine in 100 L of ethyl acetate which has been cooled to −5° C. is added to the above solution over a period of 2 hours. After the addition is complete to form a compound of formula Va, the mixture is stirred at −5° C. for another 2 hours. 23.2 kg of D-glucamine are then added a little at a time at −5° C. over a period 30 minutes and, after the addition is complete, the mixture is stirred at −5° C. for another 1 hour and subsequently at 20° C. for 12 hours. The reaction mixture is poured into 200 L of ethyl acetate and 300 L of water, the mixture is heated to 65° C., and the phases are separated. The organic phase is washed at 60° C. with a further 80 L of water and the organic phase is subsequently cooled to 20° C. over a period of 60 minutes. After stirring for another 1 hour, the precipitated solid is filtered off and dried to obtain benzyl 11-(2S,3R,4R,5R-2,3,4,5,6-pentahydroxyhexylcarbamoyl)undecanoate (I).

Subsequent experiments produced a compound of formula (II) from a compound of formula (I) under various conditions, including basic reaction conditions, enzymatic reaction conditions, and hydrogenation reaction conditions, utilizing Pd/C and hydrogen, and experiments were also performed to further produce from a compound of formula (II) a compound of formula (III), which has use for treating hyperlipidemia and arteriosclerosis and hypercholesterolemia as described in U.S. Pat. No. 7,205,290.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step, or steps, and all such modifications are intended to be within the scope of the claims appended hereto.

1-13. (canceled)

14. A compound of formula V 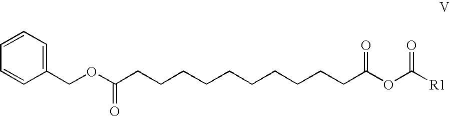
where R1 is an alkyl radical which has from 1 to 18 carbon atoms and in which at least one —CH₂— group of the alkyl radical is optionally replaced by at least one group chosen from —O— —CO—, —CH═CH—, —C≡C—, and aryl groups and the alkyl radical is optionally substituted by at least one halogen chosen from F, Cl, Br, and I.

15. A compound of formula Va

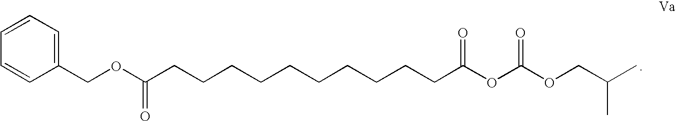

16. A compound of claim 14 of formula VIII

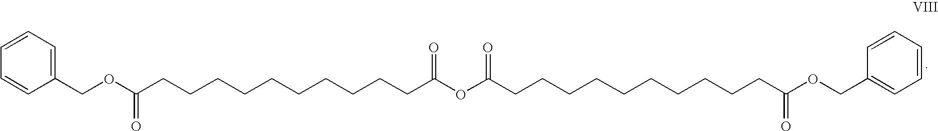

17. A process of preparing a compound of formula I 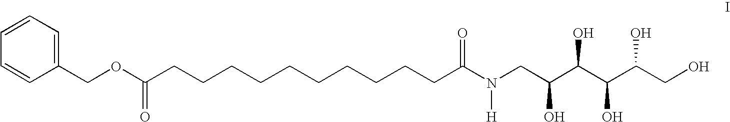
comprising converting a compound of formula V 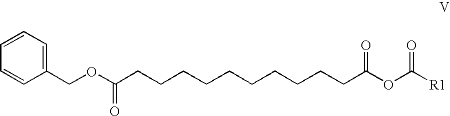
to the compound of formula I,
where R1 is an alkyl radical which has from 1 to 18 carbon atoms and in which at least one —CH₂— group of the alkyl radical is optionally replaced by at least one group chosen from —O—, —CO—, —CH═CH—, —C≡C—, and aryl groups and the alkyl radical is optionally substituted by at least one halogen chosen from F, Cl, Br, and I.

18. The process of claim 17 wherein the compound of formula V 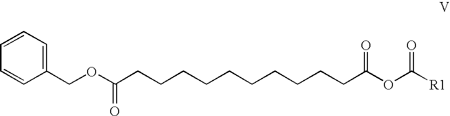
is prepared by a process comprising
reacting a compound of formula IV 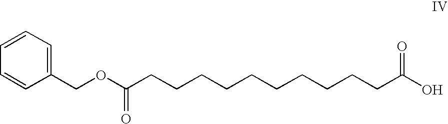
with a compound of formula VI 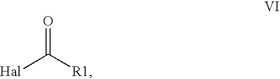
wherein
Hal is chosen from Br, Cl, and I.

19. The process of claim 18, wherein Hal is Cl.

20. The process of claim 17, wherein R1 is chosen from methyl, ethyl, propyl, isopropyl, butyl, isobutyl, tert-butyl, methyloxy, ethyloxy, propyloxy, isopropyloxy, butyloxy, isobutyloxy, tert-butyloxy, and benzyloxy.

21. The process of claim 20, wherein R1 is isopropyloxy.

22. The process of claim 17, wherein converting the compound of formula V 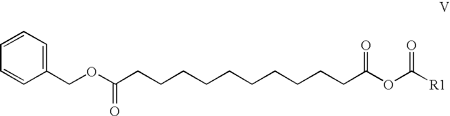
to the compound of formula I 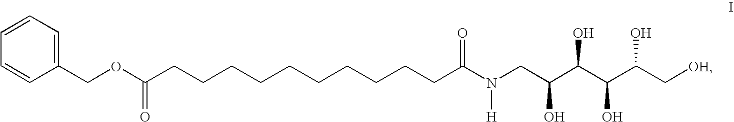
comprises reacting the compound of formula V with D-glucamine to form the compound of formula I.

23. The process of claim 17, further comprising converting the compound of formula I 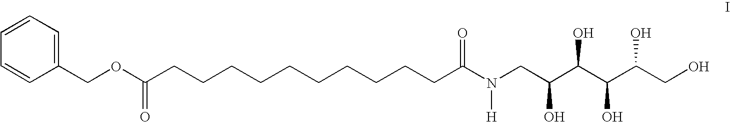
into a compound of formula II

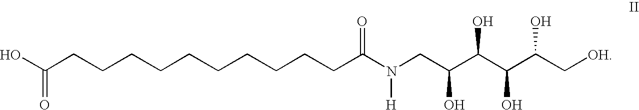

24. A process for preparing a compound of formula I 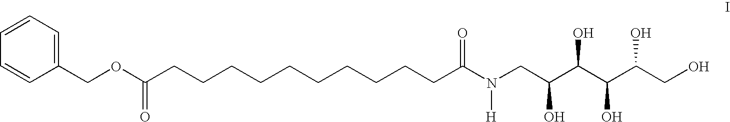
comprising converting a compound of formula Va 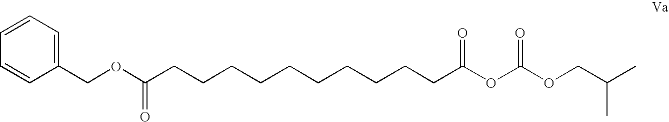
to the compound of formula I.

25. The process of claim 24, wherein the compound of formula Va 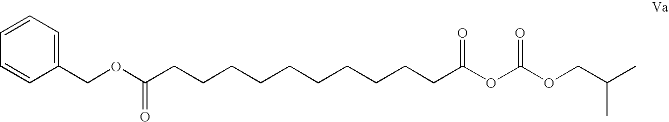
is prepared by a process comprising
reacting a compound of formula IV 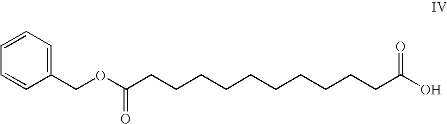
with a compound of formula VIa 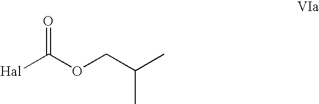
wherein Hal is chosen from Br, Cl, and I to form the compound of formula Va.

26. The process of claim 25, wherein Hal is Cl.

27. The process of claim 24, wherein converting the compound of formula Va 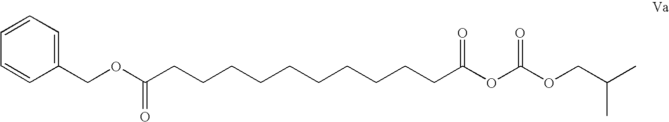
to the compound of formula I 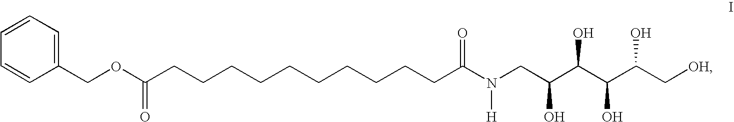
comprises reacting the compound of formula Va with D-glucamine to form the compound of formula I.

28. The process of claim 24, further comprising converting the compound of formula I into a compound of formula II

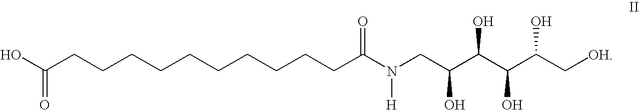

29. A process for preparing a compound of formula I 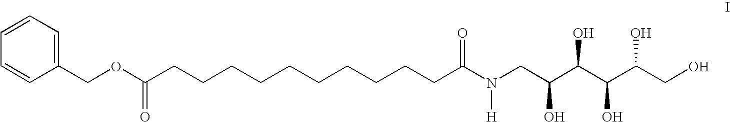
comprising converting a compound of formula VIII 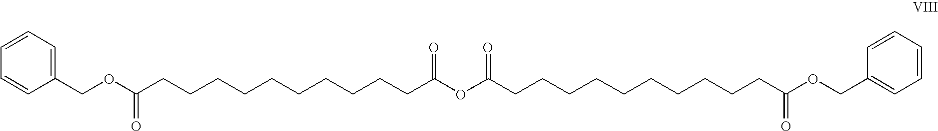
to the compound of formula I.

30. The process of claim 29 wherein the compound of formula VIII 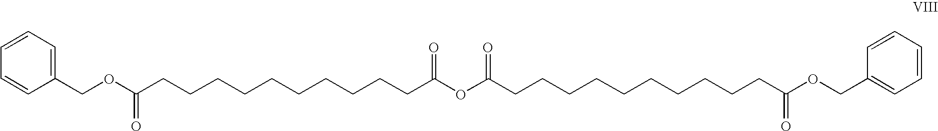
is prepared by a process comprising
a) reacting a compound of formula IV 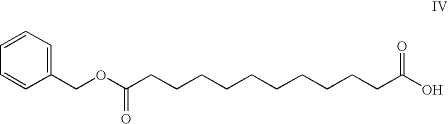
with a compound of formula VIa 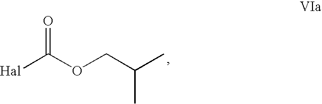
wherein Hal is chosen from Br, Cl, and I,
to form a compound of formula Va 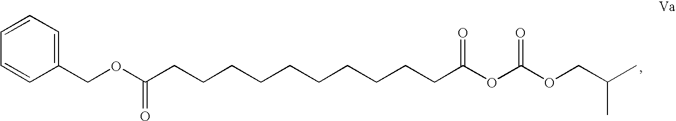
and
b) reacting the compound of formula Va with additional compound of formula IV to form the compound of formula VIII.

31. The process of claim 30, wherein Hal is Cl.

32. The process of claim 29 wherein converting the compound of formula VIII 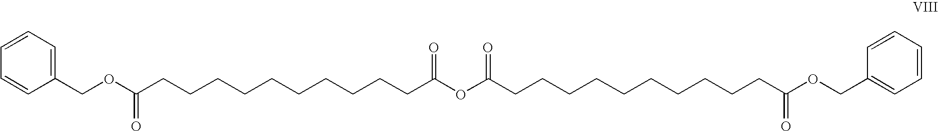
to the compound of formula I 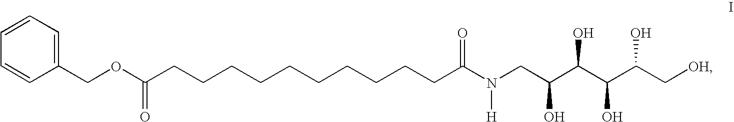
comprises reacting the compound of formula VIII with D-glucamine to form the compound of formula I.

33. The process of claim 29, further comprising converting the compound of formula I 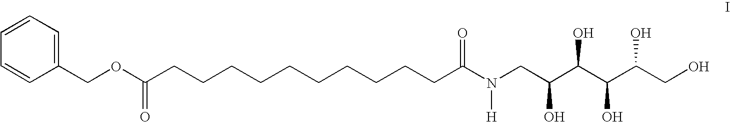
into a compound of formula II [chemical structure]
